SEQUENCE LISTING (5 sequences)

SEQ ID NO: 1 - protein, 396 aa, Plasmodium falciparum
MNYCKTTFHIFFFVLFFITIYEIKCQLRFASLGDWGKDTKGQILNAKYFKQFIKNERVTFIVSPGSNFIDGVKGLNDPAWKNLYEDVYSEEKGDMYMPFFTVLGTRDWTGNYNAQLLKGQGIYIEKNGETSIEKDADATNYPKWIMPNYWYHYFTHFTVSSGPSIVKTGHKDLAAAFIFIDTWVLSSNFPYKKIHEKAWNDLKSQLSVAKKIADFIIVVGDQPIYSSGYSRGSSYLAYYLLPLLKDAEVDLYISGHDNNMEVIEDNDMAHITCGSGSMSQGKSGMKNSKSLFFSSDIGFCVHELSNNGIVTKFVSSKKGEVIYTHKLNIKKKKTLDKVNALQHFAALPNVELTDVPSSGPMGNKDTFVRVVGTIGILIGSVIVFIGASSFLSKNMK

SEQ ID NO: 2 - protein, 346 aa, Plasmodium falciparum
KCQLRFASLGDWGKDTKGQILNAKYFKQFIKNERVTFIVSPGSNFIDGVKGLNDPAWKNLYEDVYSEEKGDMYMPFFTVLGTRDWTGNYNAQLLKGQGIYIEKNGETSIEKDADATNYPKWIMPNYWYHYFTHFTVSSGPSIVKTGHKDLAAAFIFIDTWVLSSNFPYKKIHEKAWNDLKSQLSVAKKIADFIIVVGDQPIYSSGYSRGSSYLAYYLLPLLKDAEVDLYISGHDNNMEVIEDNDMAHITCGSGSMSQGKSGMKNSKSLFFSSDIGFCVHELSNNGIVTKFVSSKKGEVIYTHKLNIKKKKTLDKVNALQHFAALPNVELTDVPSSGPMGNKDTFVR

SEQ ID NO: 3 - protein, 333 aa, Plasmodium falciparum
LRFASLGDWGKDTKGQILNAKYFKQFIKNERVTFIVSPGSNFIDGVKGLNDPAWKNLYEDVYSEEKGDMYMPFFTVLGTRDWTGNYNAQLLKGQGIYIEKNGETSIEKDADATNYPKWIMPNYWYHYFTHFTVSSGPSIVKTGHKDLAAAFIFIDTWVLSSNFPYKKIHEKAWNDLKSQLSVAKKIADFIIVVGDQPIYSSGYSRGSSYLAYYLLPLLKDAEVDLYISGHDNNMEVIEDNDMAHITCGSGSMSQGKSGMKNSKSLFFSSDIGFCVHELSNNGIVTKFVSSKKGEVIYTHKLNIKKKKTLDKVNALQHFAALPNVELTDVPSSG

SEQ ID NO: 4 - DNA, 1191 bp, Plasmodium falciparum
atgaattattgtaaaaccacgttccatatatttttttttgtcttgttctttataaccatttatgaaataaaatgtcaactacgctttgcgtctttgggtgattggggtaaggatacaaaaggtcagatattgaatgcaaagtattttaaacaatttataaagaatgaaagagttacatttatcgttagtcctggatctaatttcattgatggagttaaaggcttaaatgatccagcatggaaaaatttatatgaagatgtttattcagaagagaaaggagatatgtacatgccattttttacagtattaggtacaagagattggacaggaaattataatgctcagttattaaaaggtcaaggaatatatatagaaaaaaatggagaaacatctattgaaaaagatgcagatgctactaattatccaaaatggattatgccaaattattggtatcattattttacacattttacagtatcaagtggaccatctatagtaaaaactggccataaagatttagctgctgcatttatttttatcgatacatgggttttatcatctaacttcccatataaaaaaatacatgaaaaagcttggaatgatttaaaatcccaattatcagttgctaaaaaaattgctgattttataatagttgttggagatcaacctatttattcatctggttattctagaggtagttcttatttagcttattatttattacctttattaaaagatgcagaagtagacttatatatttcaggacatgataataatatggaagtcattgaagataatgatatggctcatattacatgtggatctggttctatgtctcaaggtaaatcaggaatgaaaaattccaaatcactcttctttagtagtgatattggtttttgcgtacatgaattatcaaataatggtatcgttaccaaattcgtcagtagtaaaaaaggagaagttatatatacacacaaacttaatatcaagaaaaaaaaaacactagataaagttaatgccttacaacactttgctgccttacccaatgtcgaattaacagatgtcccatcaagtggacccatgggaaataaagatacctttgttagagttgtaggaaccattggtattcttataggctccgtaattgtttttattggtgcttcctcttttttatcaaaaaatatgaaataa

SEQ ID NO: 5 - DNA, 999 bp, Plasmodium falciparum
ctacgctttgcgtctttgggtgattggggtaaggatacaaaaggtcagatattgaatgcaaagtattttaaacaatttataaagaatgaaagagttacatttatcgttagtcctggatctaatttcattgatggagttaaaggcttaaatgatccagcatggaaaaatttatatgaagatgtttattcagaagagaaaggagatatgtacatgccattttttacagtattaggtacaagagattggacaggaaattataatgctcagttattaaaaggtcaaggaatatatatagaaaaaaatggagaaacatctattgaaaaagatgcagatgctactaattatccaaaatggattatgccaaattattggtatcattattttacacattttacagtatcaagtggaccatctatagtaaaaactggccataaagatttagctgctgcatttatttttatcgatacatgggttttatcatctaacttcccatataaaaaaatacatgaaaaagcttggaatgatttaaaatcccaattatcagttgctaaaaaaattgctgattttataatagttgttggagatcaacctatttattcatctggttattctagaggtagttcttatttagcttattatttattacctttattaaaagatgcagaagtagacttatatatttcaggacatgataataatatggaagtcattgaagataatgatatggctcatattacatgtggatctggttctatgtctcaaggtaaatcaggaatgaaaaattccaaatcactcttctttagtagtgatattggtttttgcgtacatgaattatcaaataatggtatcgttaccaaattcgtcagtagtaaaaaaggagaagttatatatacacacaaacttaatatcaagaaaaaaaaaacactagataaagttaatgccttacaacactttgctgccttacccaatgtcgaattaacagatgtcccatcaagtgga

(19) United States
(12) Patent Application Publication
(10) Pub. No.: US 2015/0191518 A1
(43) Pub. Date: Jul. 9, 2015

(54) NOVEL MALARIA TRANSMISSION-BLOCKING VACCINES

(71) Applicant: Fraunhofer-Gesellschaft zur Förderung der angewandten Forschung e.V, Munich (DE)

(72) Inventors: Gabriele Pradel, Aldenhoven (DE); Nina Simon, Nuernberg (DE); Rainer Fischer, Aachen (DE); Andreas Reimann, Meerbusch (DE)

(21) Appl. No.: 14/584,171

(22) Filed: Dec. 29, 2014

Related U.S. Application Data
(60) Provisional application No. 61/925,018, filed on Jan. 8, 2014.

(51) Int. Cl. C07K 14/445; A61K 39/015

(52) U.S. Cl. CPC C07K 14/445; A61K 39/015

(57) ABSTRACT

Immunogenic compositions, vaccines, constructions, host cells, and vectors that include or express one or more PfGAP50 antigen or fragment thereof. The vaccine may include an adjuvant, a pharmaceutically acceptable salt, an excipient, a preservative, a binder or a pharmaceutically acceptable liquid.

### CROSS REFERENCE TO RELATED APPLICATIONS

This invention claims benefit of priority to U.S. provisional patent application Ser. No. 61/925,018, filed Jan. 8, 2014; the entire content of which is herein incorporated by reference in its entirety.

### REFERENCE TO SEQUENCE LISTING SUBMITTED ELECTRONICALLY

The official copy of the sequence listing is submitted electronically via EFS-Web as an ASCII formatted sequence listing with file “US_Appl_{—}2014-12-01_SEQID” created on 1 Dec. 2014 and having a size of 13 Kilobytes. The sequence listing contained in this ASCII formatted document forms part of the specification and is herein incorporated by reference in its entirety.

### TECHNICAL FIELD

The invention is directed to immunogenic compositions, vaccines, constructs, host cells, and vectors that include or express one or more PfGAP50 antigen or fragment thereof

### BACKGROUND

Malaria is the most prevalent tropical disease in the world, with 200 million new cases reported each year and an annual death toll of approximately 600.000 people (WHO World Malaria Report 2011). Malaria is caused by protozoan parasites of the genus Plasmodium and transmitted by blood-feeding Anopheline mosquitoes. The recurrent replication cycles of the parasite in the human red blood cells lead to the typical symptoms of the disease, like fever and anemia, and, in severe cases, can result in organ failure. Disease treatment and control measures are undermined by the spread of drug resistance, particularly in populations of P. falciparum, the agent responsible for malaria tropica (reviewed in Greenwood et al., 2008). A vaccine to protect against malaria is hitherto not available. Because of these reasons, new strategies to support and strengthen current antimalarial measures are urgently needed. A special emphasis lies on the elimination of the sexual transmission stages of the malaria parasites, because these mediate the transmission of the parasite from the human to the mosquito and are in consequence important for the spread of the disease by the vector.

The malaria sexual phase begins with the differentiation of sexual precursor cells, the intraerythrocytic gametocytes, in the human host, which are taken up by the blood-feeding mosquito (reviewed in Pradel, 2007; Kuehn and Pradel, 2010). When entering the mosquito midgut together with the blood meal, the gametocytes become activated by external stimuli, i.e. a drop in temperature and the contact with the content of the mosquito midgut.

Gametocyte activation leads to parasite egress from the enveloping erythrocyte and the formation of female macrogametes as well as male motile microgametes. Within approximately 1 hour, fertilization has occurred and the resulting zygote transforms into an infective ookinete within one day, which leaves the midgut lumen and continues the life cycle inside the mosquito (reviewed in Pradel, 2007; Kuehn and Pradel, 2010).

The sexual stages are the only life-cycle stages of the malaria parasite that are able to establish an infection in the mosquito and thus they play an important role for spread of the tropical disease. They are considered bottleneck stages with an approximate 1000-fold loss in parasite abundance during transmission to the mosquito. Noteworthy, the gametes and zygotes are the only stages within the parasite life cycle that have to persevere outside a host cell for more than one day. During this time period, the cells are highly vulnerable to the aggressive factors of the midgut, which besides digestive enzymes include human immune cells and complement proteins present in the blood meal. Because of these reasons, the malaria parasite sexual stages represent prime targets for transmission-blocking vaccines (TBVs) (reviewed in Pradel, 2007).

TBVs are target parasite antigens that are exposed in the mosquito midgut, thereby relying on human antibodies to inhibit parasite development within the mosquito host. TBVs either cause a functional inhibition of sexual stage-specific antigens or result in complement-mediated destruction of the parasites. TBVs are considered altruistic vaccines, because they would not be beneficial for the infected individual, but help to protect the community. TBVs would thus be applied as a cocktail together with other types of vaccines (reviewed in Carter, 2001; Kaslow, 2002; Pradel, 2007; Sauerwein, 2007).

Particularly antigens on the surface of gametes and zygotes are considered prospective TBV candidates. During the last two decades an increasing number of surface-associated adhesion proteins of the P. falciparum sexual stages were identified and characterized as potential TBV targets (reviewed in Pradel, 2007; Saul 2007). For example, Pfs25 was previously investigated as a TBV candidate in phase 1 clinical trials and the sera of immunized volunteers exhibited moderate transmission-blocking activity ex vivo (Wu et al., 2008).

However, there is a need for novel and more effective TBVs.

### SUMMARY OF THE INVENTION

The invention includes immunogenic compositions, vaccines, constructs, host cells, and vectors that include or express one or more PfGAP50 antigens or fragments thereof The vaccine may include an adjuvant, a pharmaceutically acceptable salt, an excipient, a preservative, a binder or a pharmaceutically acceptable liquid.

In one aspect, the invention relates to an immunogenic composition to combat the spread of malaria by using TBVs, which prevent the development of malarial parasites within its mosquito vector; thereby abrogating the cascade of secondary infections in humans. The TBV contains the Plasmodium falciparum gliding-associated protein 50 (PfGAP50), in particular at least a fragment thereof.

Some embodiments pertain to an immunogenic composition which primes an immune response in a human capable of inhibiting development of Plasmodium falciparum within a mosquito comprising an isolated PfGAP50 protein of Plasmodium falciparum or a fragment thereof.

Another aspect of the invention provides methods of priming an immune response in a human, the method including administering to a human patient an immunogenic composition according to the disclosure, thereby priming an immune response in the patient capable of inhibiting the development of Plasmodium falciparum within a mosquito.

In a further aspect, embodiments of the invention provide a recombinant vector including a nucleic acid molecule encoding the PfGAP50 polypeptide or a fragment thereof as described and to a host cell transformed, transduced or transfected with the vector.

In still another aspect, embodiments of the invention provide methods of producing a recombinant PfGAP50 polypeptide or a fragment thereof including the steps of: (a) culturing a host cell according to the invention in a suitable culture medium under suitable conditions to produce polypeptides; (b) obtaining the produced polypeptides, and optionally (c) processing the polypeptides.

In a further aspect, the invention includes transmission blocking vaccines against malaria including a recombinant virus encoding the PfGAP50 polypeptide of Plasmodium falciparum or a fragment thereof, in an amount sufficient to induce transmission blocking activity, and a pharmaceutically acceptable carrier.

### BRIEF DESCRIPTION OF THE DRAWINGS

FIG. 1 is a scheme of an isolated PfGAP50 polypeptide.

FIG. 2 is a diagram showing the dose-dependent binding of factor H (FH) and FHL-1 to PfGAP50.

FIG. 3 shows the results of immunofluorescence assays for the surface-associated expression of PfGAP50 in emerged macrogametes.

FIG. 4 is an immunoblot analysis showing Factor H-binding to the sexual stage surface in the presence of anti-PfGAP50 antisera.

FIG. 5 shows an amino acid sequence of Plasmodium falciparum gliding-associated protein 50 (SEQ ID NO: 1).

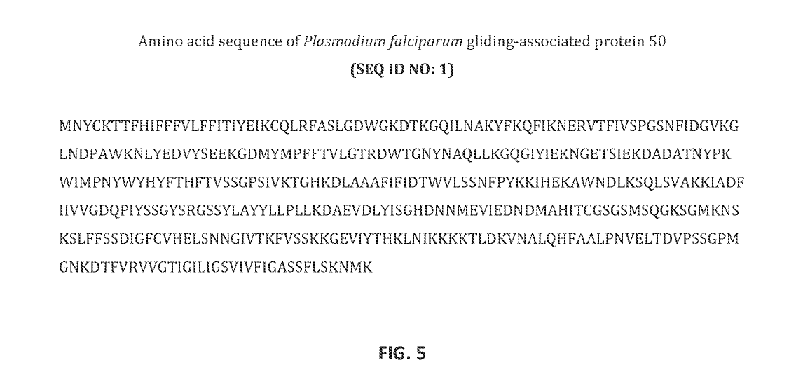

FIG. 6 shows an amino acid sequence of the PfGAP50 fragment (AAs) 24-369 (SEQ ID NO: 2).

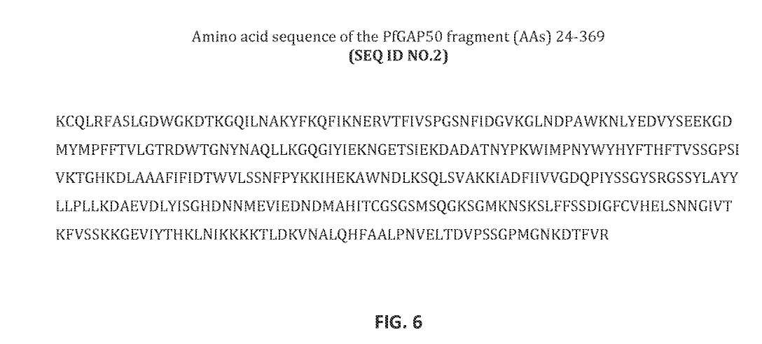

FIG. 7 shows an amino acid sequence of the PfGAP50 fragment (AAs) 27-359 (SEQ ID NO: 3).

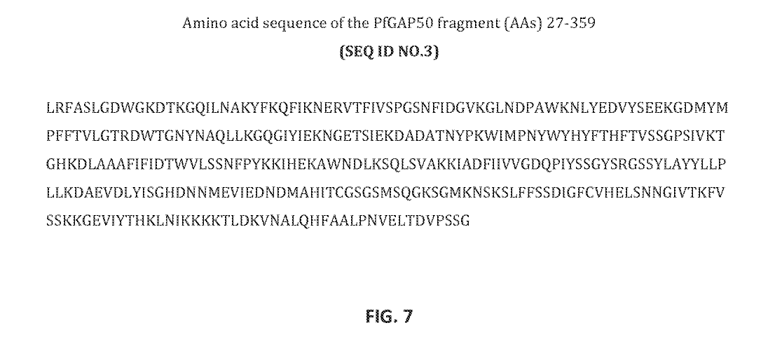

FIG. 8 shows a nucleic acid sequence encoding the Plasmodium falciparum gliding-associated protein 50 (SEQ ID NO: 4).

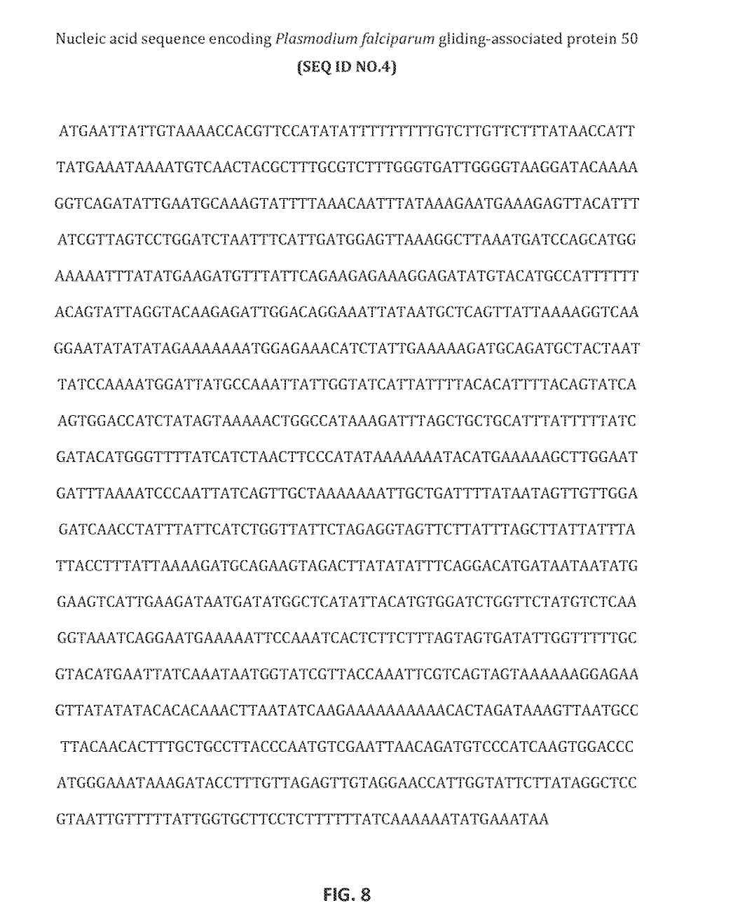

FIG. 9 shows a nucleic acid sequence encoding the AA27-359 fragment of PfGAP50 (SEQ ID NO: 5).

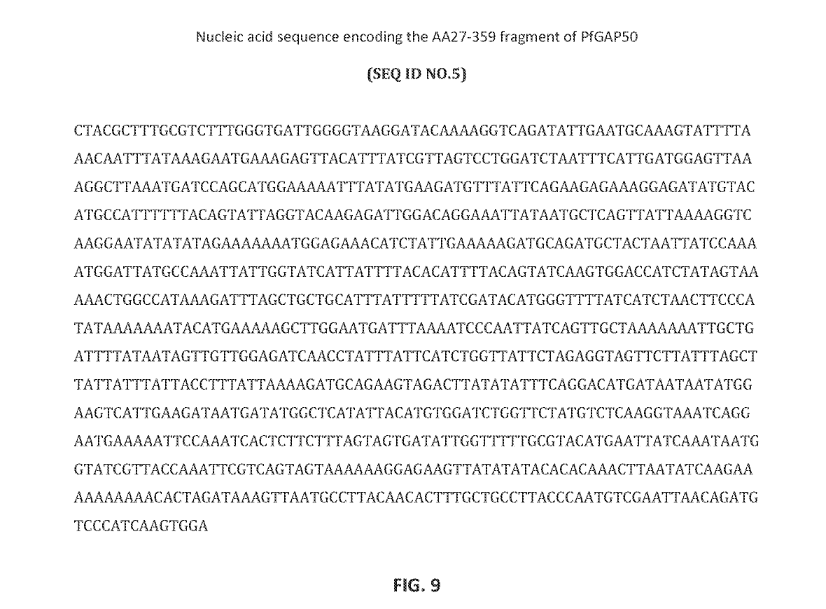

### DETAILED DESCRIPTION

Before the disclosure is described in detail, it is to be understood that the terminology used herein is for purposes of describing particular embodiments only, and is not intended to be limiting. It must be noted that, as used in the specification and the appended claims, the singular forms “a,” “an” and “the” include singular and/or plural reference unless the context clearly dictates otherwise. It is moreover to be understood that, in case parameter ranges are given which are delimited by numeric values, the ranges are deemed to include these limitation values.

The invention relates to TBVs against the tropical disease malaria. Vaccines and/or other immunogenic compositions of the invention include the Plasmodium falciparum gliding-associated protein 50 (PfGAP50) or fragments thereof, in particular vaccines comprising a polypeptide containing the amino acid sequence of SEQ ID NO: 1 or fragments thereof containing the amino acids (AAs) 24-369 (SEQ ID NO: 2) or (AAs) 27-359 (SEQ ID NO: 3), or homologous polypeptides thereof.

The term “fragment” as used herein refers to a continuous part of a natural full-length protein, with or without mutations, which is separate from and not in the context of a full length PfGAP50 surface protein. It may be a structural/topographical or functional subunit of a full length or complete protein. For example, in some embodiments of the invention fragments having an amino acid sequence of less than 20% of the parent full-length surface protein are used.

In an advantageous embodiment, the PfGAP50 surface protein or the fragments thereof are isolated polypeptides. The term “isolated” when used in relation to a nucleic acid or protein (e. g. an protein domain), refers to a nucleic acid sequence or protein that is identified and separated from at least one contaminant (nucleic acid or protein, respectively) with which it is ordinarily associated in its natural source. Isolated nucleic acid or protein is present in a form or setting that is different from that in which it is found in nature. In contrast, non-isolated nucleic acids or proteins are found in the state they exist in nature.

In some advantageous embodiments, the PfGAP50 fragments contain at least one folded domain. The term “folded domain” as used herein refers to a protein structure composed of amino acids that are arranged in a three dimensional formation. A folded domain is part of a structural/topographical or functional subunit of a full length or complete protein. It may be kept within the context of the full length or complete protein, or may be separated therefrom, as in an isolated domain. Domains corresponding to structural/topographical subunits include for example, a cytoplasmic domain, an extracellular domain or a transmembrane domain. Domains corresponding to functional subunits include for example, a receptor-binding domain or in particular an antibody binding domain.

In some advantageous embodiments, the folded domains in the PfGAP50 fragments according to the invention include at least one conformational epitope. The term “epitope” as used herein refers to a region of a protein molecule to which an antibody can bind.

The inventors have discovered that the emerging gametes of P. falciparum bind the complement regulator factor H (FH) following transmission to the mosquito to protect from complement-mediated lysis by the blood meal (Simon et al., 2013). The human complement system is active in the mosquito midgut for approximately 1 h post-feeding. During this time period, the parasite recruits FH and uses surface-bound FH to inactivate the complement cascade. The present inventors identified the plasmodial transmembrane protein PfGAP50 on the gamete surface as an FH-binding receptor. Loss of FH-mediated protection results in significantly impaired gametogenesis and inhibited parasite transmission to the mosquito. The inventors thus showed that PfGAP50 can function as a candidate for TBVs, since antibodies against PfGAP50 would functionally inhibit FH-mediated complement evasion of P. falciparum, resulting in destruction of the malaria parasite by human complement of the blood meal. Noteworthy, the unique feature of TBV antibodies against PfGAP50 is that they functionally inhibit the FH receptor PfGAP50, thereby triggering the killing of the sexual stage parasites by the human complement. One of the advantages and specific features of the use of PfGAP50 or fragments thereof as TBVs is that by binding of antibodies against PfGAP50 not only the classical complement pathway is activated, but by inhibiting FH-mediated protection, additionally the alternative pathway of human complement is stimulated. This is based on the fact that the majority of TBV antibodies act against the sexual stages of the malaria parasites together with the classical pathway of human complement (initiated by complement factor C1q) and binding of these antibodies to the sexual stage surface results in formation of a membrane attack complex and the eventual lysis of the parasite (e.g. Healer et al., 1997; Scholz et al., 2008). In the case of antibodies against GAP50, the binding of the antibodies to the receptor would on the one hand result in the above-described lysis, but would additionally block the FH-receptor, thus impairing complement evasion of the parasite against the alternative pathway of complement (initiated by complement factor C3b). The alternative pathway, since not inhibited any longer, will also result in the formation of a membrane attack complex and lysis of the parasite.

Therefore, PfGAP50 is an excellent TBV, and in particular fragments of PfGAP50 can be used as a vaccine for preventing the transmission of the parasites that cause malaria. Once an immune response against PfGAP50 is stimulated in a subject, preferably a person, the antibodies produced against the PfGAP50 are effective to disrupt the development of the parasites.

In other embodiments, the TBVs of the invention can be used with currently available vaccine for malaria, including those disclosed in WO 2006/088597, WO 2006/088597, and WO 2008/009650, which are hereby incorporated by reference. The TBV can be administered concurrently with the malaria vaccine. Alternatively, the TBV can be admixed with the malaria vaccine to produce a combination vaccine and administered as a single dose.

In some embodiments, the invention shows that bacterially produced peptides comprising AA27-359 of PfGAP50 (SEQ ID NO: 3) elicit the production of transmission-blocking antibodies in mice. In some examples, a gene of a recombinant fragment of PfGAP50 has been engineered by chemical synthesis and mutagenesis, and the gene as expressed in Escherichia coli to produce a recombinant fragment of PfGAP50, designated rPfGAP50. Absent from rPfGAP50 was the amino-terminal secretory sequence and amino- and carboxyl terminal trans membrane domain sequences. rPfGAP50 bound recombinant human FH in standard-binding assays. Further, mice inoculated with rPfGAP50 have developed antibodies which i) bind to the surface of the midgut-specific sexual stages of P. falciparum (particularly female macrogametes), ii) reduce the binding of FH to the surface of the sexual stage parasites in in vitro assays, and iii) block sexual development of P. falciparum in the mosquito vector in ex vivo assays. Anopheles stephensi mosquitoes, which were ex vivo fed with Plasmodium falciparum blood cultures containing mature gametocytes in the presence of the PfGAP50-specific mouse antisera showed reduced infection rates compared to mosquitoes fed with control antisera, thus the rPfGAP50-specific antisera exhibits transmission-blocking activity by interfering with binding of FH to the sexual stage surface.

Furthermore, the invention includes homologous polypeptides of the PfGAP50 polypeptide (SEQ ID NO: 1), and in particular to PfGAP50 fragments comprising the amino acid sequence of SEQ ID NO: 2 that correlates to the amino acids 24-369 sequence of SEQ ID NO: 1 and/or SEQ ID NO: 3 that correlates to the amino acids 27-359 sequence of SEQ ID NO: 1, both lacking the amino-terminal secretory signal and the transmembrane regions. In some embodiments, the encoded polypeptide may also lack one or more N-linked glycosylation sites.

As mentioned above, the invention further includes TBVs against malaria. A TBV prevents the transmission of P. falciparum from host to mosquito vector. The TBVs of the present invention can also include other malarial antigens, for examples the PfGAP50 peptides might be fused to other antigens generating protective malarial immunity.

The present invention is based on studies that involved recombinant polypeptides containing the PfGAP50 of Plasmodium falciparum or fragments thereof and the use of such polypeptides for inoculation of mice to determine immunogenicity and efficacy as a vaccine. From these studies, it was determined that PfGAP50 elicits the production of transmission-blocking antibodies.

In general, the invention pertains to TBVs against malaria comprising PfGAP50 polypeptides or variants, modified forms, homologs, fusion proteins and fragments thereof

For example, a homologous polypeptide according to the invention includes any polypeptides with a percentage sequence identity of at least 70% or preferably at least 75%, 80%, 85%, 90%, 95% or 99% to SEQ ID NO: 1, SEQ ID NO: 2 or SEQ ID NO: 3.

In an advantageous embodiment, the immunogenic composition according to the invention includes a fragment of the PfGAP50 protein having the amino acid sequence of SEQ ID NO: 2 or SEQ ID NO: 3, or a sequence having at least a minimum percent sequence identity of 75% to SEQ ID NO: 2 or SEQ ID NO: 3, in particular a sequence having at least a minimum percent sequence identity of 85% to SEQ ID NO: 2 or SEQ ID NO: 3.

“Percent sequence identity”, with respect to two amino acid or polynucleotide sequences, refers to the percentage of residues that are identical in the two sequences when the sequences are optimally aligned. Thus, 80% amino acid sequence identity means that 80% of the amino acids in two optimally aligned polypeptide sequences are identical. Percent identity can be determined, for example, by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in “Atlas of Protein Sequence and Structure”, M. O. Dayhoff et., Suppl. 3:353-358, National Biomedical Research Foundation, Washington, D.C., which adapts the local homology algorithm of Smith and Waterman (1981) Advances in Appl. Math. 2:482-489 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. An example of an algorithm that is suitable for determining sequence similarity is the BLAST algorithm, which is described in Altschul, et ah, J. MoI. Biol. 215:403-410 (1990). Software for performing BLAST analyses is publicly available through the National Center for Biotechnology Information (http://www.ncbi.nlm.nih.gov/). Likewise, computer programs for determining percent homology are also readily available.

The term “homologue of the nucleic acid molecule” refers to a nucleic acid molecule the sequence of which has one or more nucleotides added, deleted, substituted or otherwise chemically modified in comparison to a nucleic acid molecule according to one of the claimed sequences, provided always that the homologue retains substantially the same enzymatic and/or stability properties as the latter.

In further advantageous embodiments, the immunogenic compositions according to the invention further include an adjuvant and/or an immunostimulating agent like an immunostimulating nanoparticle, wherein the immunostimulating nanoparticle includes protein sequences from an animal virus, a human virus and/or a plant virus

In other embodiments, the invention relates to recombinant attenuated viruses encoding the plasmodial transmembrane protein of Plasmodium falciparum, designated PfGAP50 protein or polypeptide and/or fragments thereof The invention also relates to recombinant attenuated viruses encoding a unique portion of the PfGAP50 polypeptide, wherein a unique portion includes a polypeptide having an amino acid sequence of SEQ ID NO: 2 or SEQ ID NO: 3, or homologs thereof The viruses are attenuated using methods known in the art, for example, the method described in Bueller et al., Nature 317:813 (1985).

Suitable viruses for use in the invention include, but are not limited to, pox viruses, such as, for example, canarypox and cowpox viruses, and vaccinia viruses, alpha viruses, adenoviruses, and other animal viruses. The recombinant viruses can be produced methods well known in the art, for example, using homologous recombination or ligating two plasmids together. A recombinant canarypox or coxpox virus can be made, for example, by inserting the gene encoding PfGAP50 or a fragment thereof into a plasmid so that it is flanked with viral sequences on both sides. The gene is then inserted into the virus genome through homologous recombination.

A recombinant adenovirus virus can be produced, for example, by ligating together two plasmids each containing 50% of the viral sequence and the DNA sequence encoding PfGAP50 or a fragment thereof Recombinant RNA viruses such as the alpha virus can made via a cDNA intermediate using methods known in the art.

The recombinant virus of the present invention can be used to induce anti-PfGAP50 antibodies in mammals, such as mice or humans. In addition, the recombinant virus can be used to produce PfGAP50 protein by infecting host cells which in turn express PfGAP50.

As mentioned above, the invention includes vaccines against malaria. In particular, the invention includes a TBV. A TBV prevents the transmission of Plasmodium falciparum from host to mosquito vector.

One TBV of the invention includes a unique portion of PfGAP50 of Plasmodium falciparum and a pharmaceutically acceptable carrier. The vaccine may also include adjuvant. The vaccine can be administered via intradermal, subcutaneous, intramuscular, nasopharyngeal or respiratory routes, for example, inhalation.

Another TBV of the present invention includes Pfs25 or a unique peptide thereof, purified from host cells infected with a recombinant virus, and a pharmaceutically acceptable carrier. The protein is purified using standard purification techniques known in the art. This embodiment of the vaccine is particularly useful for booster inoculations. Mice inoculated with the recombinant virus have a relatively low antibody titer, however, when these mice are given subsequent booster inoculations of purified host cell membrane extracts their antibody titers increase as does the transmission-blocking activity of the antibodies.

In further embodiments, the TBVs of the invention can also include other malarial antigens. For example, the TBV of the invention includes antigens generating protective malarial antibodies.

The present invention also relates to methods of preventing transmission of malarial infections. Methods of the present invention comprise administering to a patient a vaccine as disclosed, in an amount sufficient to induce transmission-blocking activity. The treatment consists of a single administration or a series of administrations.

As mentioned above, one embodiment of the invention includes methods for producing a PfGAP50 polypeptide or fragments thereof including the steps of: (a) culturing a host cell having nucleic acid molecules encoding the PfGAP50 or fragments thereof in a suitable culture medium under suitable conditions to produce PfGAP50 or fragments thereof; (b) obtaining the produced polypeptides, and optionally (c) processing the polypeptides.

In order to produce the PfGAP50 polypeptide or fragments thereof, the DNA encoding the polypeptides (“PfGAP50 genes”) can be chemically synthesized from published sequences or obtained directly from host cells harboring the gene (e.g., by cDNA library screening or PCR amplification). The PfGAP50 gene can be included in an expression cassette and/or cloned into a suitable expression vector by standard molecular cloning techniques. Such expression cassettes or vectors often contain sequences that assist initiation and termination of transcription (e.g., promoters and terminators), and may contain selectable markers. Cassettes can also be comprised of plus or minus strand mRNA, and their expression may or may not include an amplification step before translation of the mRNA. The glucose oxidase gene to be expressed can contain or not contain certain domains of the protein, such as polymer binding domains (e.g., carbohydrate binding domains) of various specificities. The expression cassette or vector can be introduced in a suitable expression host cell which will then express the corresponding PfGAP50 gene. Particularly suitable expression hosts are bacterial expression host genera including Escherichia (e.g. E. coli), Pseudomonas (e.g. P. fluorescens or P. stutzerei), Proteus (e.g. Proteus mirabilis), Ralstonia (e.g. R. eutropha), Streptomyces, Staphylococcus (e.g. S. carnosus), Lactococcus (e.g. L. lactis), and Bacillus (e.g. B. subtilis, B. megaterium, B. licheniformis). Also particularly suitable are yeast expression hosts such as Saccharomyces cerevisiae, Schizosaccharomyces pombe, Yarrowia lipolytica, Hansenula polymorpha, Kluyveromyces lactis or Pichia pastoris. Especially suited are fungal expression hosts such as Chrysosporium lucknowense, Aspergillus (e.g. A. oryzae, A. niger, A. nidulans) or Trichoderma reesei. Also suited are mammalian expression hosts such as mouse (e.g. NS0), Chinese hamster ovary (CHO) or baby hamster kidney (BHK) cell lines, transgenic mammalian systems such as rabbit, goat or cattle, other eukaryotic hosts such as insect cells or plants, or viral expression systems such as bacteriophages M13, T7 or lambda, or eukaryote viruses such as Baculovirus.

PfGAP50 genes can be introduced into the expression host cells by a number of transformation methods including, but not limited to, electroporation, lipid-assisted transformation or transfection (“lipofection”), chemically mediated transfection (e.g., CaCl and/or CaP), lithium acetate-mediated transformation (e.g. of host-cell protoplasts), biolistic “gene gun” transformation, PEG-mediated transformation (e.g. of host-cell protoplasts), protoplast fusion (e.g. using bacterial or eukaryotic protoplasts), liposome-mediated transformation, Agrobacterium tumefaciens, adenovirus or other viral or phage transformation or transduction.

The protein of interest can be secreted into the extracellular or periplasmic space or expressed intracellularly. Optionally, after intracellular expression of the enzyme variants, or secretion into the periplasmic space using signal sequences such as those mentioned above, a permeabilisation or lysis step can be used to release the protein of interest into the supernatant. The disruption of the membrane barrier can be effected by the use of mechanical means such as ultrasonic waves, pressure treatment (French press), cavitation or the use of membrane-digesting enzymes such as lysozyme or enzyme mixtures. As a further alternative, the genes encoding the glucose oxidase enzyme are expressed cell-free by the use of a suitable cell-free expression system. For example, the S30 extract from Escherichia coli cells was used for this purpose or commercially available systems (e.g. CECF technology by Roche Applied Science, Inc.). In cell-free systems, the gene of interest was typically transcribed with the assistance of a promoter, but ligation to form a circular expression vector is optional. RNA can also be exogenously added or generated without transcription and translated in cell free systems. Configurations of expression constructs for in vitro expression and execution of all of the above expression systems are well within the ability of the skilled artisan.

As described above, the PfGAP50 polypeptides or fragments thereof can be expressed in a variety of expression systems and accordingly the appropriate downstream processing and purification procedures have to be selected. In an advantageous embodiment of the disclosure the PfGAP50 gene is expressed in a microbial host and the protein is secreted into the periplasmic or extracellular space. Cells expressing the PfGAP50 are preserved by methods well known to those skilled in the art, such as, but not limited, to cryo stocks. Cultures of the expressing organism are prepared at an appropriate volume with standard methods of fermentation. In a preferred embodiment, cultures for protein expression are inoculated from a cryo stock and the volume of the culture increased successively in the appropriate containers. In a preferred embodiment the cells are grown in a fermenter and optionally growth conditions such as pH, temperature, oxygen and/or nutrient supply are controlled. A first step of purification comprises the separation of cells from supernatant using one or more of several techniques, such as sedimentation, microfiltration, centrifugation, or flocculation. In a preferred embodiment the method applied is microfiltration. In case of intracellular expression the cells are subjected to treatments that result in a release of the protein from the intracellular space. These treatments may comprise for example pressure, enzymatic, osmotic shock, freezing, ultrasonic or other treatment to produce a cellular extract, which may or may not be subjected to further purification.

In an advantageous embodiment the protein is secreted into the supernatant and an optional step of purification comprises the concentration of the supernatant by ultrafiltration. Further protein purification from the supernatant or concentrated supernatant may be performed with one or more of several methods comprising extraction or fractionation methods such as ammonium sulfate or ethanol or acid precipitation, or chromatographic methods including but not limited to ion-exchange, hydrophobic interaction, hydroxylapatite, size fractionation by gel-filtration, phosphocellulose or lectin chromatography and affinity chromatography or any combination thereof In a more preferred method the affinity-tagged protein is purified by metal-chelate affinity chromatography to obtain a high purity protein.

In another advantageous embodiment the supernatant or the supernatant partially purified by ultrafiltration or the concentrated and/or diafiltrated supernatant is dried by any one of several technical methods such as, but not limited to, spray-drying, lyophilisation, down-draught evaporation, thin-layer evaporation, centrifugal evaporation, conveyer drying or any combination thereof

In a further advantageous embodiment the fermented cell-suspension including the expressed PfGAP50 or PfGAP50 fragment is dried as a whole using processes such as, but not limited to, fluidized bed drying, conveyer drying, spray drying or drum drying or any combination thereof

The polypeptide of interest that is produced may be recovered, further purified, isolated, processed and/or modified by methods known in the art. For example, the polypeptide may be recovered from the nutrient medium by conventional procedures including, but not limited to, centrifugation, filtration, ultrafiltration, extraction or precipitation. Further processing steps such as purification steps may be performed by a variety of procedures known in the art including, but not limited to, chromatography (e.g. ion exchange, affinity, hydrophobic, chromatofocusing, and size exclusion), electrophoretic procedures (e.g., preparative isoelectric focusing), differential solubility (e.g. ammonium sulfate precipitation) or extraction. Furthermore, the isolated and purified polypeptide of interest may be further processed, e.g. formulated, into a composition, in particular to a immunogenic composition like a vaccine composition.

The following examples are given to further illustrate the present invention without being deemed limitative thereof.

### EXAMPLES

In the following examples, materials and methods of the invention are provided. It should be understood that these examples are for illustrative purpose only and are not to be construed as limiting this disclosure in any manner. All publications, patents, and patent applications cited herein are hereby incorporated by reference in their entirety for all purposes.

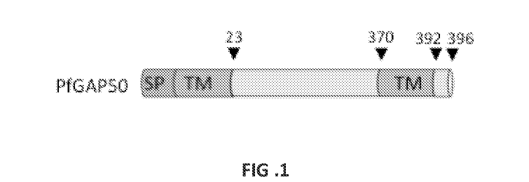

A PfGAP50 gene (SEQ ID NO: 5) encoding AA27-359 of the natural 396 AA of the PfGAP50 protein was chemically synthesized using methods previously disclosed (FIG. 1; Simon et al., 2013). The synthesized gene lacked the amino-terminal secretory sequence of PfGAP50 protein and the amino- and carboxyl-terminal transmembrane regions, as predicted in (Bosch et al., 2012). In FIG. 1 the selected AA positions are indicated. TM is the abbreviation for transmembrane domain; SP is the abbreviation for signal peptide.

Expression and secretion from E. coli BL21(DE3)RIL cells of the synthesized rPfGAP50 peptide was achieved by fusion of the synthetic gene to DNA sequences encoding for a GST (glutathione S transferase), using the commercial vector pGEX 4T-1 (Amersham Biosciences) as described (Simon et al., 2013).

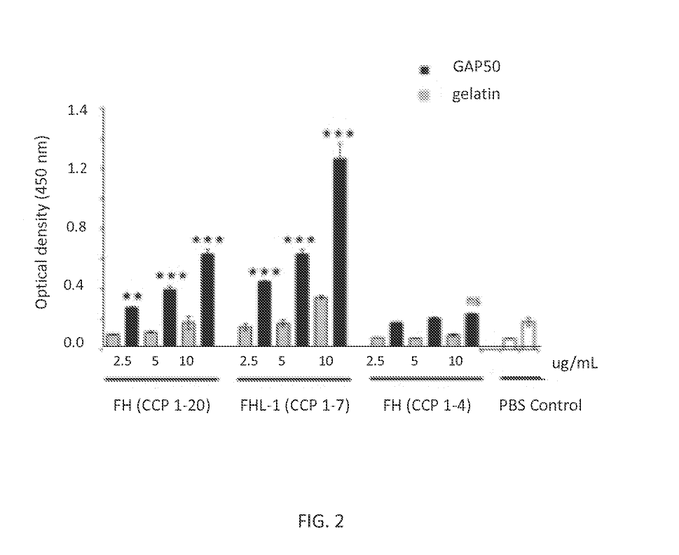

Protein rPfGAP50 was purified via immobilized glutathione according to the manufacturer's protocol (Amersham Biosciences). rPfGAP50 or gelatine as control (10 μg/ml) were coated onto MaxiSorp microtiter plates (Nunc) for 4° C. overnight and incubated with equimolar amounts of FH (comprising complement control modules CCP1-20) or fragments thereof (CCP1-7=FHL-1, or CCP1-4; each 2.5, 5, or 10 μg/ml) The FH peptides were recombinantly expressed in Pichia pastoris as described (Skerka et al., 2007; Weismann et al., 2011). Bound proteins were detected with polyclonal FH antiserum (CompTech Tyler) and secondary horseradish conjugated rabbit anti-goat antibody. The reaction was developed with 1,2 phenylenediamine dihydrochloride (Dako-Cytomation) and absorbance was measured at OD (450 nm). The standard-binding assay showed that FH and fragment CCP1-7 specifically bind to rPfGAP50 in a dose-dependent manner (FIG. 2). FIG. 2 is a diagram showing the dose-dependent binding of FH and FHL-1 to PfGAP50. Recombinant immobilized PfGAP50 was incubated with recombinant CCP1-20 (=FH), CCP1-7 (=FHL-1) and CCP1-4 at concentrations of 2.5, 5, and 10 μg/ml. FH peptide binding was measured colorimetrically at OD (450 nm). Immobilized gelatine and PBS alone were used for negative controls. Significant differences in protein binding are indicated (*p<0.05, **p<0.01, ***p<0.001; student's t-test). ns, not significant (from Simon et al., 2013).

For the generation of immunsera, rPfGAP50 was isolated as inclusion bodies as described (Scholz et al., 2008). Specific immune sera were generated by the immunization of 6-week-old female NMRI mice (Charles River Laboratories) with 100 μg recombinant protein emulsified in Freund's incomplete adjuvant (Sigma-Aldrich) followed by a boost after 4 weeks. Mice were anesthetized by intraperitoneal injection of a mixture of ketamine and xylazine according to the manufacturer's protocol (Sigma-Aldrich), and immune sera were collected 10 days after the second immunization via heart puncture.

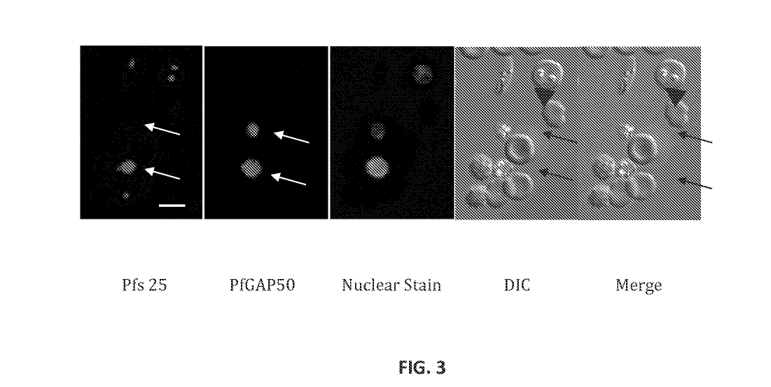

The binding of PfGAP50-specific mouse antisera was demonstrated by indirect immunofluorescence assay as described (Simon et al., 2013). P. falciparum gametocytes were activated by 100 μM xanthurenic acid for 15 min. The cultures were washed in PBS and then fixed in 4% PFA/PBS in suspension for 2 h at 4° C. The fixed cells were subsequently blocked in 3% BSA in PBS for 30 min and incubated for 1.5 h at 37° C. with the PfGAP50-specific mouse antisera diluted in 0.5% BSA/PBS. Binding of primary antibody was visualized using Alexa Fluor 488-conjugated goat anti-mouse antibodies (Molecular Probes). Subsequently, specimens were incubated with the respective second antibody against the macrogamete-specific surface protein Pfs25 followed by Alexa Fluor 594-conjugated goat anti-rabbit secondary antibody. The cells were finally stained with Hoechst 33342 (Invitrogen) according to the manufacturer's protocol. Labeled specimens were examined by a Live Imaging Leica AF 6000 microscope and showed that PfGAP50 is expressed on the surface of female macrogametes. The indirect immunofluorescence assays revealed that PfGAP50 is present on the surface of the emerged macrogametes and here recognized by the PfGAP50-specific antisera (FIG. 3). FIG. 3 shows the surface-associated expression of PfGAP50 in emerged macrogametes. Gametocytes (GC) were activated, fixed with PFA at 30 min p.a. and subjected indirect immunofluorescence assays without saponin-permeabilization. PfGAP50 was labeled with polyclonal anti-PfGAP50 antisera (green), macrogametes were highlighted by Pfs25-labeling (red). Nuclei were highlighted with Hoechst nuclear stain (blue). Bar, 5 um (modified from Simon et al., 2013).

The reduced binding of FH to the sexual stage surface in the presence of PfGAP50-specific mouse antisera was demonstrated by cell-binding assays and Western blot analysis as described (Simon et al., 2013). Gametocytes were activated by 100 μM xanthurenic acid in the absence of human serum and in the presence of polyclonal mouse anti-PfGAP50 antisera or neutral mouse antiserum, which was used as a negative control (a 10% (v/v) concentration was used). At 15 min p a , FH was added to the activated gametocytes and the cells were incubated for another 15 min. The gametocyte lysates were separated by gel electrophoresis, followed by Western blot analysis, and probed with anti-FH antisera. Western blotting showed that blocking of PfGAP50 by the respective antisera resulted in reduced binding of

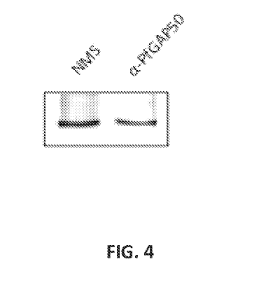

FH to the activated gametocytes, when compared to neutral mouse serum (FIG. 4). FIG. 4 shows the FH-binding to the sexual stage surface in the presence of anti-PfGAP50 antisera. Gametocytes were activated with xanthurenic acid in the presence of mouse anti-PfGAP50 antisera or neutral mouse serum and FH was subsequently added. Parasite pellets were harvested and subjected to gel-electrophoresis and Western blotting, using anti-FH antiserum. Similar results were obtained, when the activated gametocytes were incubated with PfGAP50-specific rabbit antisera (Jones et al., 1996) prior to the addition of FH. Again, the binding of FH to the sexual stage surface was reduced compared to controls incubated with a control rabbit control antiserum directed against the plasmodial protease plasmepsin II (Simon et al., 2013).

The transmission-blocking effect of the PfGAP50-specific mouse antisera was demonstrated by transmission-blocking assays (Simon et al., 2013). An. stephensi mosquitoes were fed with a 5% sterile saccharose solution supplemented with para-aminobenzoic acid (PABA) and 40 μg/ml gentamicin until two days prior to the assay (Beier et al., 1994). Percoll-purified gametocytes were mixed with human serum containing FH, fresh human erythrocytes and the antisera at 37° C. Mouse anti-PfGAP50 as well as anti-Pf39 antiserum or neutral mouse antiserum (NMS, both used as controls) were tested at concentrations of 10% (v/v). The mixture was offered to 3-5 day old female An. stephensi mosquitoes via glass feeders and the mosquitoes were allowed to feed for 20 min (Bishop and Gilchrist, 1946). Mosquitoes with engorged midguts were collected, and at 10 d post-feeding, the midguts of these mosquitoes were dissected and stained with 0.2% mercurochrome in PBS. The numbers of oocysts per midgut were counted under the microscope. Three independent experiments were performed. Mosquitoes fed on gametocyte cultures in the presence of NMS exhibited infection rates of 47% and 50%, and mosquitoes fed in the presence of anti-Pf39 antisera showed an infection rate of 40%. The TBAs revealed that the presence of anti-PfGAP50 antibody reduced the transmission rates by 40%, 68% and 38%, when compared to the respective controls (TABLE 1), demonstrating that anti-GAP50 antisera blocked the transmission of P. falciparum to the mosquito.

TABLE 1

| | | # | | | Infec- | |
|---|---|---|---|---|---|---|
| | | inf./ | # | oocysts/ | tion | Reduction |
| | Feed | total | range | midgut, | rate | in transm. |
| TBA 1 | NMS | 10/20 | 3 ± 3, 0-9 | | 50% | |
| | α-PfGAP50 | 6/20 | 2 ± 1, 0-2 | | 30% | 40% |
| TBA 2 | NMS | 8/17 | 1 ± 1, 0-3 | | 47% | |
| | α-PfGAP50 | 3/20 | 1 ± 1, 0-2 | | 15% | 68% |
| TBA 3 | α-Pf39 | 8/20 | 2 ± 1, 0-4 | | 40% | |
| | α-PfGAP50 | 5/20 | 1 ± 1, 0-2 | | 25% | 38% |

TBA, transmission-blocking assay; NMS, neutral mouse serum.

### REFERENCES

The contents of all cited references, including literature references, issued patents, and published patent applications, as cited throughout this application are hereby expressly incorporated by reference.

Beier M S, Pumpuni C B, Beier J C, Davis J R (1994) Effects of para-aminobenzoic acid, insulin, and gentamicin on Plasmodium falciparum development in anopheline mosquitoes (Diptera: Culicidae). J Med Entomol 31, 561-565.

Bishop A, Gilchrist B M (1946) Experiments upon the feeding of Aedes aegypti through animal membranes with a view to applying this method to the chemotherapy of malaria. Parasitology 37, 85-100.

Bosch J, Paige M H, Vaidya A B, Bergman L W, Hol W G (2012) Crystal structure of GAP50, the anchor of the invasion machinery in the inner membrane complex of Plasmodium falciparum. J Struct Biol 178, 61-73.

Carter R (2001) Transmission blocking malaria vaccines. Vaccine 19, 2309-2314.

Greenwood B M, Fidock D A, Kyle D E, Kappe S H, Alonso P L, Collins F H, Duffy P E (2008) Malaria: progress, perils, and prospects for eradication. J Clin Invest 118, 1266-1276.

Healer J, McGuinness D, Hoperoft P, Haley S, Carter R, Riley E (1997) Complement-mediated lysis of Plasmodium falciparum gametes by malaria-immune human sera is associated with antibodies to the gamete surface antigen Pfs230. Infect Immun 65, 3017-3023.

Kaslow DC (2002) Transmission-blocking vaccines. Chem Immunol 80, 287-307.

Kuehn A, Pradel G (2010) The coming-out of malaria gametocytes. J Biomed Biotechnol 2010, 976827.

Pradel G (2007) Proteins of malaria parasite sexual stages: expression, function and potential for transmission blocking strategies. Parasitology 134, 1911-1929.

Sauerwein R W (2007) Malaria transmission-blocking vaccines: the bonus of effective malaria control. Microbes Infect 9, 792-795.

Saul A (2007) Mosquito stage, transmission blocking vaccines for malaria. Curr Opin Infect Dis 20, 476-481.

Scholz S M, Simon N, Lavazec C, Dude M A, Templeton T J, Pradel G (2008) PfCCp proteins of Plasmodium falciparum: gametocyte-specific expression and role in complement-mediated inhibition of exflagellation. Int J Parasitol 38, 327-340.

Simon N, Scholz S M, Moreira C, Templeton T J, Kuehn A, Dude M A, Pradel G (2009) Sexual stage adhesion proteins form multi-protein complexes in the malaria parasite Plasmodium falciparum. J Biol Chem 284, 14537-14546.

Simon N, Lasonder E, Scheuermayer M, Kuehn A, Tews S, Fischer R, Zipfel P F, Skerka C, Pradel G. (2013) Malaria parasites co-opt human factor H to prevent complement-mediated lysis in the mosquito midgut. Cell Host Microbe 13, 29-41.

Skerka C, Lauer N, Weinberger A A, Keilhauer C N, Sühnel J, Smith R, Schlotzer-Schrehardt U, Fritsche L, Heinen S, Hartmann A, Weber B H, Zipfel P F.(2007) Defective complement control of factor H (Y402H) and FHL-1 in age-related macular degeneration. Mol Immunol 44, 3398-3406.

Weismann D, Hartvigsen K, Bennett K L, Cano M, Brandstätter H, Tsimikas S, Skerka C, Superti-Furga G, Handa J T, Zipfel P F, et al. (2011) Complement factor H binds malondialdehyde-epitopes and protects from oxidative stress. Nature 478, 76-81.

Wu Y, Ellis R D, Shaffer D, Fontes E, Malkin E M, Mahanty S, Fay M P, Narum D, Rausch K, Miles A P, Aebig J, Orcutt A, Muratova O, Song G, Lambert L, Zhu D, Miura K, Long C, Saul A, Miller L H, Durbin A P (2008) Phase 1 trial of malaria transmission blocking vaccine candidates Pfs25 and Pvs25 formulated with montanide ISA 51. PLoS One 3, e2636.

What is claimed is:

1. An immunogenic composition that primes an immune response in a human, capable of inhibiting development of Plasmodium falciparum within a mosquito, wherein the composition comprises an isolated PfGAP50 polypeptide of Plasmodium falciparum or a fragment thereof

2. The immunogenic composition according to claim 1, wherein the isolated PfGAP50 polypeptide comprises the amino acid sequence of SEQ ID NO: 1, or a sequence having at least a minimum percent sequence identity of 75% to SEQ ID NO: 1.

3. The immunogenic composition according to claim 1, wherein the fragment of the isolated PfGAP50 polypeptide comprises the amino acid sequence of SEQ ID NO: 2, or a sequence having at least a minimum percent sequence identity of 75% to SEQ ID NO: 2.

4. The immunogenic composition according to claim 1, wherein the fragment of the isolated PfGAP50 polypeptide comprises the amino acid sequence of SEQ ID NO: 3, or a sequence having at least a minimum percent sequence identity of 75% to SEQ ID NO: 3.

5. The immunogenic composition according to claim 1, wherein the PfGAP50 polypeptide or the fragment thereof is a recombinant polypeptide.

6. The immunogenic composition according to claim 1, further comprising an adjuvant and/or an immunostimulating agent, optionally an immunostimulating nanoparticle, wherein the optional immunostimulating nanoparticle comprises protein sequences from a virus selected from one or more of the group consisting of an animal virus, a human virus and a plant virus.

7. The immunogenic composition according to claim 1, further comprising a protective malarial antigen.

8. A method of priming an immune response in a human, the method comprising administering to a human patient a composition according to claim 1 , thereby priming an immune response in the patient capable of inhibiting the development of Plasmodium falciparum within a mosquito.

9. The method according to claim 8, wherein the composition is administered to the patient via a route selected from the group consisting of intradermally, subcutaneously, intramuscularly, nasopharyngeally, and a respiratory route.

10. A transmission blocking vaccine against malaria comprising a recombinant virus encoding the PfGAP50 polypeptide of Plasmodium falciparum or a fragment thereof, in an amount sufficient to induce transmission blocking activity, and a pharmaceutically acceptable carrier.

11. The vaccine according to claim 10, wherein the PfGAP50 protein comprises the amino acid sequence of SEQ ID NO: 1, or a sequence having at least a minimum percent sequence identity of 75% to SEQ ID NO: 1.

12. The vaccine according to claim 10, wherein the fragment of the PfGAP50 polypeptide comprises the amino acid sequence of SEQ ID NO: 2, or a sequence having at least a minimum percent sequence identity of 75% to SEQ ID NO: 2.

13. The vaccine according to claim 10, wherein the fragment of the PfGAP50 polypeptide comprises the amino acid sequence of SEQ ID NO: 3, or a sequence having at least a minimum percent sequence identity of 75% to SEQ ID NO: 3.

14. The vaccine according to claim 10, wherein the virus is selected from the group consisting of an alpha virus, cowpox virus, canarypox virus, adenovirus, vaccinia virus, an other animal virus, and an other human virus.
